(19) United States
(12) Patent Application Publication
(10) Pub. No.: US 2025/0077044 A1
(43) Pub. Date: Mar. 6, 2025

(54) SYSTEMS AND METHODS OF PERSONALIZED, SEMI-AUTOMATIC FEATURE ANALYSIS FOR MEDICAL IMAGING

(71) Applicant: KONINKLIJKE PHILIPS N.V., EINDHOVEN (NL)

(72) Inventors: Hyeonwoo Lee, Cambridge, MA (US); Goutam Ghoshal, South Grafton, MA (US); Mohsen Zahiri, Cambridge, MA (US); Balasundar Iyyavu Raju, North Andover, MA (US)

(21) Appl. No.: 18/818,976

(22) Filed: Aug. 29, 2024

Related U.S. Application Data
(60) Provisional application No. 63/535,374, filed on Aug. 30, 2023.

(51) Int. Cl. G06F 3/0482; G06F 3/04847; G06F 3/0488; G06T 7/00; G16H 30/40; G16H 40/63

(52) U.S. Cl. CPC G06F 3/0482; G06F 3/04847; G06F 3/0488; G06T 7/0012; G16H 30/40; G16H 40/63; G06T 2200/24; G06T 2207/20104; G06T 2207/30004

(57) ABSTRACT

The present disclosure is directed to systems and methods that address and improve upon certain technical challenges arising from the increasing use of artificial intelligence (AI) in medical imaging analysis. More specifically, the systems and methods described herein provide personalized, semi-automatic feature analysis that streamlines decision-making and workflow, simplifies the selection and use of available AI tools, and improves the functionality of these AI tools by enabling user input as key stages of the analysis.

### CROSS-REFERENCE TO PRIOR APPLICATIONS

This application claims the benefit of Provisional Application No. 63/535,374, filed Aug. 30, 2023, the contents of which are herein incorporated by reference.

### GOVERNMENT INTEREST

This invention was made with United States government support awarded by the United States Department of Health and Human Services under the grant number HHS/ASPR/BARDA 75A50120C00097. The United States has certain rights in this invention.

### FIELD OF THE DISCLOSURE

The present disclosure relates generally to systems and methods of medical imaging analysis, and more specifically to systems and methods of personalized, semi-automatic feature analysis for medical imaging. The systems and methods described herein may find particular application with point-of-care ultrasound imaging devices.

### BACKGROUND

In the field of medical imaging, artificial intelligence (AI) is increasingly used to help a user find important features in particular images, such as ultrasound images. For example, AI applications may allow users to mark or tag important features that can then be used for quality analysis, medical diagnosis, and education purposes. Two common operations in the field of medical image analysis include object detection and feature segmentation. As used herein, the term “object detection” is used to describe the method of localizing features in a medical image. In certain examples, an AI prediction algorithm may present its object detection prediction by generated a bounding box around a potential feature. The term “segmentation,” on the other hand, localizes and includes all the pixels that belong to the features in the image. That is, segmentation provides more information since it localizes and highlights the actual region of the features.

Deep learning-based AI models are the state-of-the-art method to perform these tasks object detection and segmentation. However, training an accurate and reliable segmentation model is more difficult compared to training an accurate and reliable detection model. Acquiring a good annotation for segmentation requires much more time and effort from medical professionals. Additionally, segmentation slows the inference speed which makes it difficult to perform real-time analysis.

Furthermore, determining whether to employ AI models for object detection and/or segmentation depends on a number of factors, including the target features, the user's level of expertise, and the user's personal preference. For typical features in the abdomen region such as the liver, spleen, and kidney, a medical doctor might prefer to have object detection with a bounding box only. On the other hand, for features in the suprapubic regions such as bladder or free fluid, segmentation may be preferred. In some instances, a highly-trained expert may tend to prefer bounding boxes over segmentation since they can see the actual features without overlaying with the mask or outline, whereas a novice user may prefer segmentation that highlights the boundary of the features and makes it easier to interpret. In further instances, personal preference may play a strong role when deciding which form of model prediction should be used.

Accordingly, the increasing use of AI tools in medical imaging analyses has the effect of making a one-size-fits-all workflow approach unlikely to be successful for a number of reasons.

### SUMMARY OF THE DISCLOSURE

The present disclosure is directed to systems and methods of personalized, semi-automatic feature analysis for medical imaging, which provide a flexible workflow when using AI model predictions for medical image analysis. As described herein, the systems and methods enable a user to readily choose between feature segmentation and object detection, and to selectively adjust parameters related to the AI model prediction at various stages in the analysis. As a result, the systems and methods described herein provide a flexible workflow that also improves the results of the AI model predictions by enabling user guidance at key stages. Although the present disclosure may find particular application with point-of-care ultrasound settings, it should be appreciated that other imaging modalities may also benefit from the systems and methods described herein, including but not limited to magnetic resonance imaging (MRI) and computed tomography (CT).

According to an embodiment of the present disclosure, a system configured to assist in the medical imaging analysis of a subject is provided. The system may include an electronic device comprising: a display device configured to display an image analysis interface; a user input device configured to receive user input from a user; a computer-readable storage medium having stored thereon machine-readable instructions to be executed by one or more processors; and one or more processors configured by the machine-readable instructions stored on the computer-readable storage medium to perform the following operations: (i) receive imaging data of the subject; (ii) generate and display on the display device an image analysis interface, wherein the image analysis interface comprises the imaging data and a menu of user-selectable options; (iii) receive user input via the user input device, wherein the user input includes a selection of one or more trained artificial intelligence models; (iv) analyze the imaging data based on the user input received.

In an aspect, the one or more trained artificial intelligence models can include at least one of a trained object localization model and a trained feature segmentation model.

In an aspect, the one or more processors can be further configured to receive user input via the user input device, wherein the user input includes an input parameter used to modify the operation of the one or more trained artificial intelligence models.

In an aspect, the input parameter may include a region of interest within at least one medical image generated based on the received imaging data of the subject.

In an aspect, the image analysis interface generated by the one or more processors and displayed on the display device may further include an output of the one or more trained artificial intelligence models.

In an aspect, the output of the one or more trained artificial intelligence models can include at least one of: a bounding box identifying a region within at least one medical image generated based on the received imaging data of the subject, wherein the region contains an anatomical feature of the subject identified by the one or more trained artificial intelligence models; a bounding polygon segmenting an anatomical feature within at least one medical image generated based on the received imaging data of the subject, wherein the segmented anatomical feature was identified by the one or more trained artificial intelligence models; and a classification of an anatomical feature within at least one medical image generated based on the received imaging data of the subject, wherein the anatomical feature was identified by the one or more trained artificial intelligence models.

In an aspect, the menu of user-selectable options of the image analysis interface may include: a first option to select a first trained artificial intelligence model from the one or more trained artificial intelligence models, wherein the first trained artificial intelligence model is a trained object localization model; a second option to select a second trained artificial intelligence model from the one or more trained artificial intelligence models, wherein the second trained artificial intelligence model is a trained feature segmentation model; a third option to modify an output of at least one of the first and second trained artificial intelligence models; and a fourth option to provide an input parameter used to modify the operation of at least one of the first and second trained artificial intelligence models.

In an aspect, the display device can be a touch-enabled display, the user input device can include the touch-enabled display, and the user input can comprise touch data received via the touch-enabled display.

In an aspect, the system may further comprise a medical imaging device in communication with the electronic device and configured to obtain imaging data of the subject, wherein the medical imaging device includes at least one of an ultrasound imaging device, a magnetic resonance imaging machine, and a computed tomography machine.

According to another embodiment of the present disclosure, a point-of-care imaging system configured to assist in the medical imaging analysis of a subject is provided. The system may include a medical imaging device configured to obtain imaging data of the subject, and an electronic device in communication with the medical imaging device, wherein the electronic device comprises: a display device configured to display an image analysis interface; a user input device configured to receive user input from a user; a computer-readable storage medium having stored thereon machine-readable instructions to be executed by one or more processors; and one or more processors configured by the machine-readable instructions stored on the computer-readable storage medium to perform the following operations: (i) receive imaging data of the subject from the medical imaging device; (ii) generate and display on the display device an image analysis interface, wherein the image analysis interface comprises the imaging data and a menu of user-selectable options; (iii) analyze the received imaging data using at least a first trained artificial intelligence model, wherein an output of the first trained artificial intelligence model includes a bounding box identifying a region within at least one medical image generated based on the received imaging data, and wherein the region includes an anatomical feature of the subject identified by the first trained artificial intelligence model; (iv) receive user input via the user input device, wherein the user input includes an input parameter used to modify the operation of at least a second trained artificial intelligence model; and (v) analyze the received imaging data using at least the second trained artificial intelligence model based on the input parameter received as user input.

In an aspect, the first trained artificial intelligence model may be a trained object localization model and the second trained artificial intelligence model may be a trained feature segmentation model.

In an aspect, the input parameter received as user input may include an adjustment of the output of the first trained artificial intelligence model.

According to still another embodiment of the present disclosure, a computer-implemented method for personalized, semi-automatic feature analysis using a medical imaging system is provided. In an aspect, the medical imaging system can include a medical imaging device and an electronic device in communication with the medical imaging device. The method can include: obtaining, via the medical imaging device, imaging data of a subject; displaying, on a display device of the electronic device, an image analysis interface comprising one or more images generated based on the obtained imaging data, and a menu of user-selectable options; receiving, via a user input device of the electronic device, user input including a selection of at least a first trained artificial intelligence model, wherein at least the first trained artificial intelligence model is selected from the menu of user-selectable options; in response to receiving the selection of at least the first trained artificial intelligence model, analyzing the obtained imaging data using at least the first trained artificial intelligence model; updating the image analysis interface displayed via the display device of the electronic device to include an output of at least the first trained artificial intelligence model selected from the menu of user-selectable options; receiving, via the user input device of the electronic device, user input including an input parameter used to modify the operation of one or more trained artificial intelligence models; receiving, via the user input device of the electronic device, user input including a selection of at least a second trained artificial intelligence model selected from the menu of user-selectable options; in response to receiving the selection of at least the second trained artificial intelligence model, analyzing the obtained imaging data using at least the second trained artificial intelligence model and the input parameter received as user input; and updating the image analysis interface displayed via the display device of the electronic device to include an output of at least the second trained artificial intelligence model.

In an aspect, the input parameter received as user input can include an adjustment of the output of at least the first trained artificial intelligence model.

In an aspect, the first trained artificial intelligence model may be a trained object localization model, and the second trained artificial intelligence model may be a trained feature segmentation model.

These and other aspects of the various embodiments will be apparent from and elucidated with reference to the embodiments described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a diagram of a point-of-care imaging system configured to assist in the medical imaging analysis in accordance with aspects of the present disclosure.

FIG. 2 is an illustration of an electronic device and image analysis interface shown in accordance with aspects of the present disclosure.

FIG. 3 is an illustration of an image analysis interface shown in accordance with aspects of the present disclosure.

FIG. 4 is an illustration of an image analysis interface shown in accordance with further aspects of the present disclosure.

FIG. 5 is an illustration of an image analysis interface shown in accordance with further aspects of the present disclosure.

FIG. 6 is an illustration of an image analysis interface shown in accordance with further aspects of the present disclosure.

FIG. 7 is an illustration of an image analysis interface shown in accordance with further aspects of the present disclosure.

FIG. 8 is a schematic diagram illustrating the components of a point-of-care imaging system in accordance with aspects of the present disclosure.

FIG. 9 is a flowchart illustrating a computer-implemented method for personalized, semi-automatic feature analysis in accordance with aspects of the present disclosure.

### DETAILED DESCRIPTION OF EMBODIMENTS

The present disclosure is directed to systems and methods for personalized, semi-automatic medical image analysis that enables cross-functionality between multiple, independent artificial intelligence (AI) models. As described above, AI is increasingly used in the field of medical imaging to help users find important features in particular images, such as ultrasound images. However, determining whether the deploy a particular AI model or type of AI model often depends on a number of factors. For example, a seasoned healthcare professional may have a preferred workflow and require less intensive support from AI tools, whereas a less experienced user may be reassured making diagnostic determinations with the support for additional AI tools.

Additionally, different AI tools may not be configured to automatically work together. For example, an AI model dedicated to one function (e.g., object localization) may not interact with another AI model dedicated to a different function (e.g., feature segmentation). As a result, running multiple AI tools without express need can be computationally expensive and can negatively impact the usefulness of these technical solutions in the real world.

According to the present disclosure, the systems and methods described enable a user to select one or more AI tools (i.e., trained AI models) and provide user input modifying the operation of these AI tools, thereby personalizing the imaging analysis workflow and reducing unnecessary expenditures in terms of time and computational power. As such, the systems and methods described herein may find particular application in real-time analysis of medical imaging data (i.e., analysis performed concurrently or substantially concurrently with the imaging itself), such as in point-of-care ultrasound imaging. However, it should be appreciated that other imaging modalities may be used, including but not limited to magnetic resonance imaging (MRI) and computed tomography (CT).

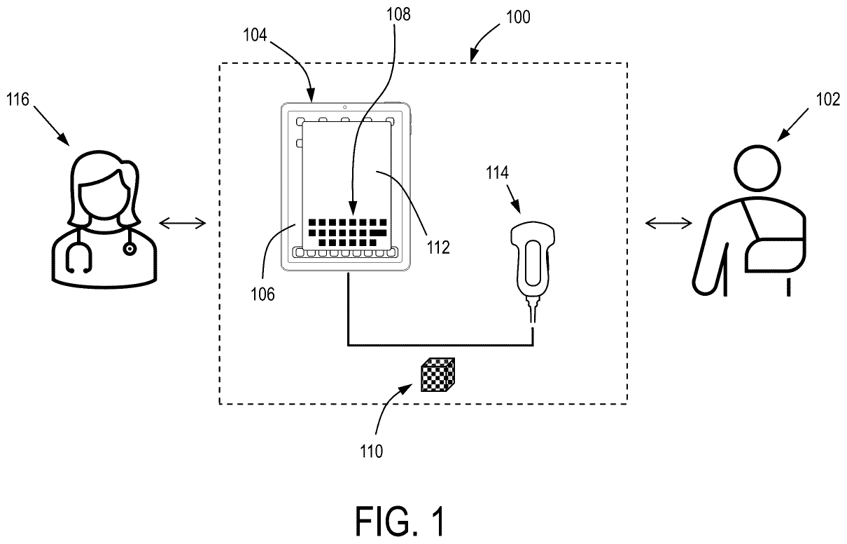

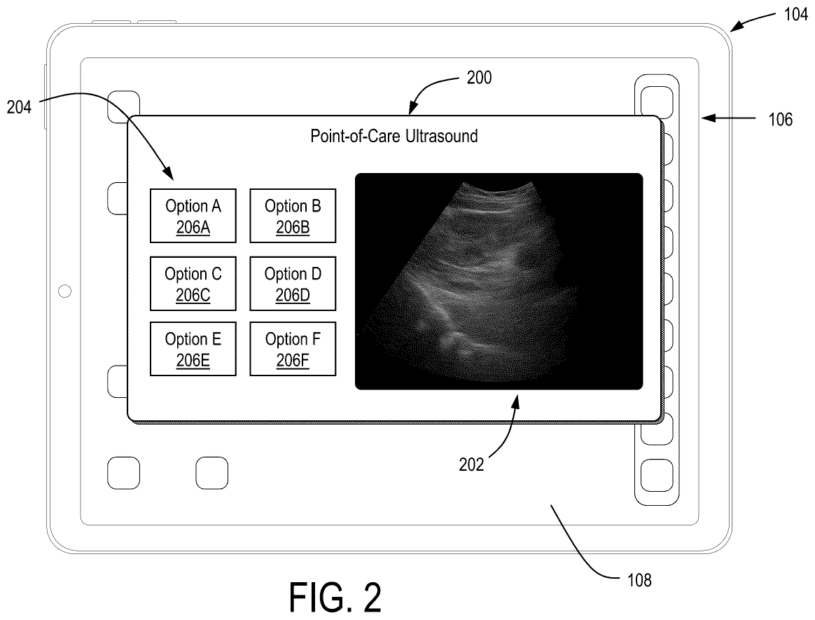

Turning now to FIG. 1, a medical imaging analysis system 100 configured to assist in the medical imaging analysis of a subject 102 is illustrated in accordance with various aspects of the present disclosure. As shown, the system 100 may include an electronic device 104 configured to perform one or more operations of the methods described herein. In particular embodiments, the electronic device 104 may include one or more of the following: a display device 106, a user input device 108, a computer-readable storage medium (e.g., memory *** as shown in FIG. 2), and one or more processors (e.g., processors *** as shown in FIG. 2). In embodiments, the one or more processors of the electronic device 104 may be configured by machine-readable instructions to perform at least some of the following operations: one or more processors configured by the machine-readable instructions stored on the computer-readable storage medium to perform the following operations: (i) receive imaging data 110 of the subject 102; (ii) generate and display on the display device 106 an image analysis interface 112, wherein the image analysis interface 112 comprises the imaging data 110 and a menu of user-selectable options; (iii) receive user input via the user input device 108, wherein the user input includes a selection of one or more trained artificial intelligence models; and (iv) analyze the imaging data 110 based on the user input received.

In embodiments, the medical imaging analysis system 100 may also include a medical imaging device 114. The medical imaging device 114 may be operatively connected to and/or otherwise in communication with the electronic device 104, as shown in FIG. 1. In embodiments, the medical imaging device 114 can be configured to obtain imaging data 110 of the subject 102. In particular embodiments, the medical imaging device 114 can be an ultrasound probe, a magnetic resonance imaging (MRI) machine, a computed tomography (CT) machine, and/or the like. As such, in various embodiments, the imaging data 110 of the subject 102 can include ultrasound imaging data, MRI scan data, CT scan data, and/or the like. In embodiments, the imaging data 110 may be received by the electronic device 104 in a “raw” format and processed by the electronic device 104 into one or more medical images. Alternatively, the imaging data 110 may be preprocessed into one or more images prior to being received by the electronic device 104. That is, the electronic device 104 may receive imaging data 110 in an unprocessed or processed format.

As described herein, the medical imaging analysis system 100 may be a portable system and/or a point-of-care system configured to perform the medical imaging of the subject 102 and the real-time imaging analysis. Put another way, in some embodiments, the medical imaging analysis system 100 may be a point-of-care imaging system configured to assist in the medical imaging analysis of a subject 102 in real-time (i.e., the imaging analysis may be performed concurrently or substantially concurrently with the imaging itself). Accordingly, in particular embodiments, the medical imaging device 114 may be reversibly or detachably coupled to the electronic device 104 such that the device 114 may be connected and disconnected from the electronic device 104 as needed.

In embodiments, the display device 106 of the electronic device 104 may be configured to display an image analysis interface 110 in accordance with various aspects of the present disclosure. For example, the display device 106 may be a liquid crystal display (LCD), a light-emitting diode (LED) display, a touch screen or other touch-enabled display, a foldable display, a projection display, and so on, or combinations thereof.

In embodiments, the user input device 108 may be configured to receive user input from a user 116. For example, the user input device 108 may include a peripheral device, such as one or more of a keyboard, keypad, trackpad, trackball(s), capacitive keyboard, controller (e.g., a gaming controller), computer mouse, computer stylus/pen, a voice input device, and/or the like, including combinations thereof. In particular embodiments, the display device 106 and the user input device 108 may be the same device, such as a touch-enabled display. In such embodiments, for example, the user input can include touch data received via the touch-enabled display.

In particular embodiments, the user input can include a selection of one or more trained artificial intelligence models, which may be used to analyze the imaging data 110. For example, in some embodiments, the one or more trained artificial intelligence models can include at least one of a trained object localization model and a trained feature segmentation model. In further embodiments, the user input can include an input parameter used to modify the operation of one or more such trained artificial intelligence models. For example, the user input may specify a particular size or location relative to the imaging data 110 for analysis. That is, the user input may identify a region of interest within the medical imaging data 110 that is used to limit or focus the imaging analysis by the one or more trained artificial intelligence models.

In still further embodiments, the input parameter received as a user input can include an adjustment of the output of one or more previously executed trained artificial intelligence models. For example, in embodiments, a trained object localization model may be used to analyze the imaging data 110 and output coordinates specifying a region within the imaging data 110 where a feature of interest is likely present. The output of such a trained artificial intelligence model may be expressed in a number of ways, including a set of (x,y) coordinates, or visually as a bounding box over a particular image generated based on the imaging data 110. Thus, the input parameter received as a user input can include, for example, an adjustment to the (x,y) coordinates and/or the bounding box defining a region within the imaging data 110.

Although certain input parameters have been discussed herein, it is contemplated that other types of input parameters may be provided as user input and used to correct, specify, and/or modify the analysis of a subsequent trained artificial intelligence model. For example, in some embodiments, the input parameters can also include, but are not limited to, a class of instance (e.g., when an AI model incorrectly identifies a subject's liver as the subject's bladder, the user can provide feedback/correction).

In specific embodiments, the system 100 is configured to enable a semi-automatic feature detection and segmentation workflow with a combination of two or more different artificial intelligence models and user input in a point-of-care imaging setting. These systems 100 address several technical challenges in conventional systems. For example, in conventional approaches, the user cannot personalize their preferences on segmentation versus object detection, the segmentation operation is limited to the feature inside of the predicted bounding box which can lead to under-segmentation, and the segmentation operation can lead to over-segmentation if the predicted bounding box is larger than the actual feature size. Additionally, instance segmentation is computationally expensive and therefore leads to slow inference time, which limits the usefulness of conventional systems in actual practice.

As described herein, the system 100 may generate and display on a display device 106 an image analysis interface for interacting dynamically with the obtained imaging data 110. For example, with reference to FIG. 2, an electronic device 104 having a display device 106 displaying an image analysis interface 200 in accordance with certain aspects of the present disclosure. As shown, the electronic device 104 also includes a user input device 108 that is a touch-enabled screen. Put another way, the display device 106 and the user input device 108 are a touchscreen. The electronic device 104 and the image analysis interface 200 provides a personalized and versatile workflow that can fit into different groups of users, provides various options to see the predictions of different AI tools, allow the enabling/disabling of certain annotations and/or predictions, and allow the user to provide input parameters for different AI tools.

In the example of FIG. 2, the image analysis interface 200 can include imaging data 110 of one or more subjects (e.g., subject 102), including one or more medical images 202 generated based on the imaging data 110. In embodiments, the image analysis interface 200 can also include a menu 204 of user-selectable options 206A-206F that are configured to enable the user to provide certain types of input or perform certain types of operations.

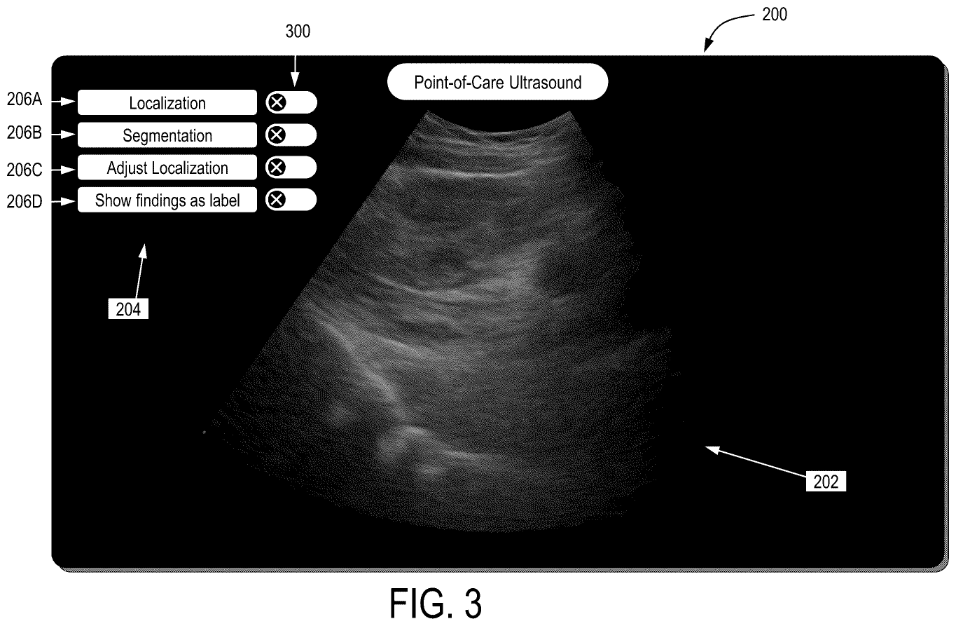

For example, with reference to FIG. 3, another image analysis interface 200 including a medical image 202 of a subject 102 and a menu 204 of user-selectable options 206A-206D is shown in accordance with specific aspects of the present disclosure. In particular, the user-selectable options include: a first option 206A to apply a first trained artificial intelligence model to the imaging data 110 (i.e., the medical image 202); a second option 206B to apply a second trained artificial intelligence model to the imaging data 110 (i.e., the medical image 202); a third option 206C to modify an output of at least one of the trained artificial intelligence models; and a fourth option 206D to display the findings of one or more trained artificial intelligence models as a label. As shown, the menu 204 may include one or more indicators 300 corresponding to the options 206A-206D that indicate whether a user-selectable feature 206A-206D has been enabled.

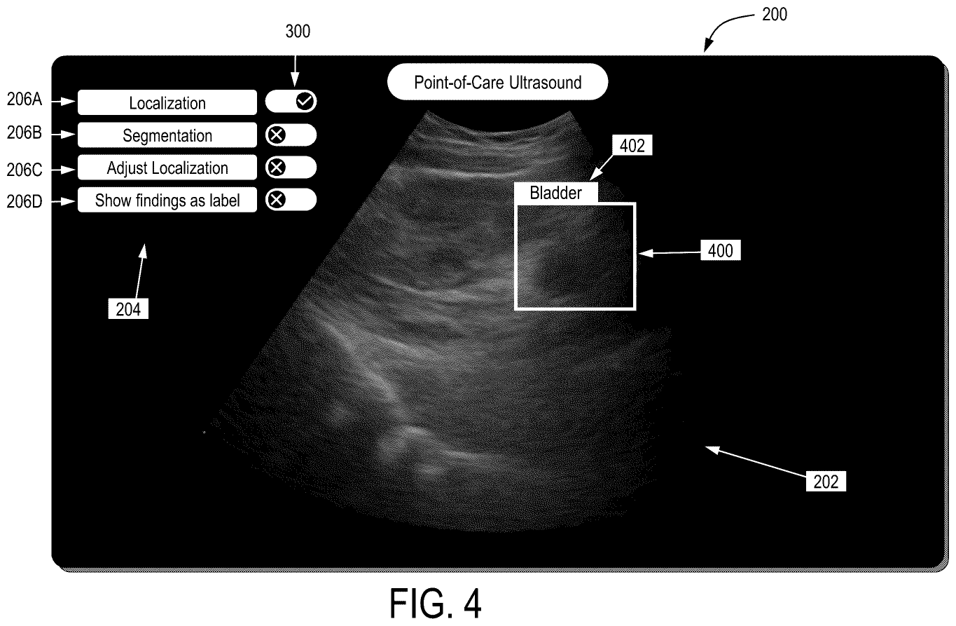

With reference to FIGS. 4-7, the features and workflow enabled by the systems 100 of are illustrated in accordance with various aspects of the present disclosure. In the example of FIG. 4, the first option 206A is selected by the user 116 and a first trained artificial intelligence model is applied to the imaging data 110, wherein the trained artificial intelligence model is an object localization model trained to identify and localize a plurality of anatomical features in the imaging data 110. As shown, the output of the trained model can include a bounding box 400 identifying a region within at least one medical image 202 generated based on the received imaging data 110 of the subject 102, wherein the region contains or exhibits an anatomical feature of the subject 102 that was identified by one or more trained artificial intelligence models. In embodiments, the output of the object localization model may also include a set of (x,y) coordinates identifying the region(s) within at least one medical image 202 generated based on the imaging data 110 where an anatomical feature of the subject 102 is present. In further embodiments, the output of the localization model may also include a classification of one or more anatomical features identified based on the imaging data 110. For example, as shown in FIG. 4, the anatomical feature identified is the subject's bladder, which may be represented in the image analysis interface 200 as a feature label 402.

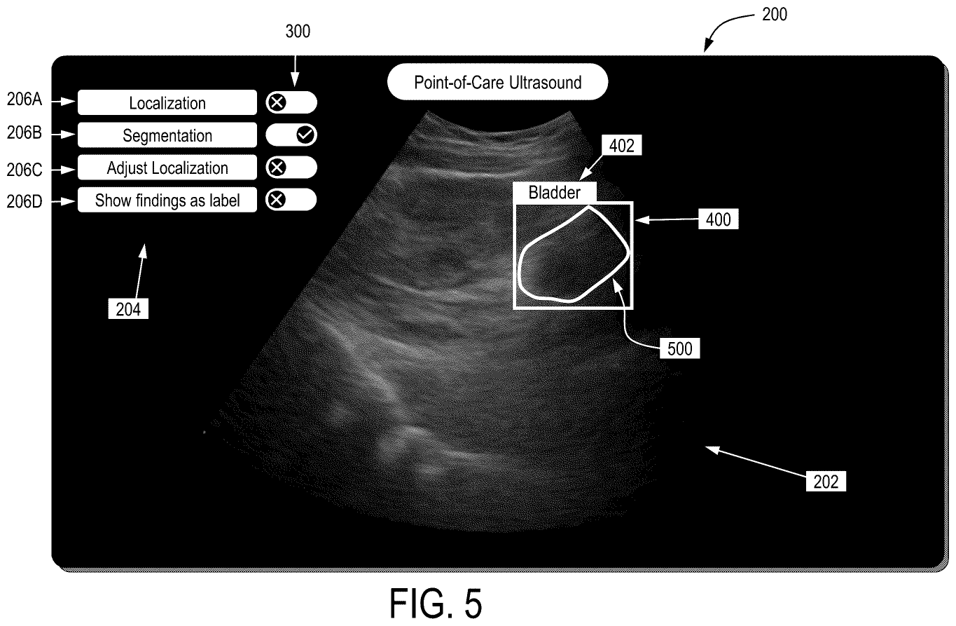

In the example of FIG. 5, an image analysis interface 200 is illustrated where the second option 206B is selected by the user 116 and a second trained artificial intelligence model is applied to the imaging data 110. In particular, the second trained artificial intelligence model can be a feature segmentation model trained to identify, localize, and segment one or more anatomical features based on the imaging data 110 of the subject 102. As shown, the output of the segmentation model may include a bounding box 400 identifying a region where an anatomical feature is present, and a bounding polygon 500 predicting the borders/edges of the anatomical feature. The output of the segmentation model may be stored as a digital mask or masking layer generated by the segmentation model, and may be represented visually as a bounding polygon 500, as shown in FIG. 5. In embodiments, the output may also include a classification of one or more anatomical features segmented by the segmentation model, which may be represented visually as a label 402.

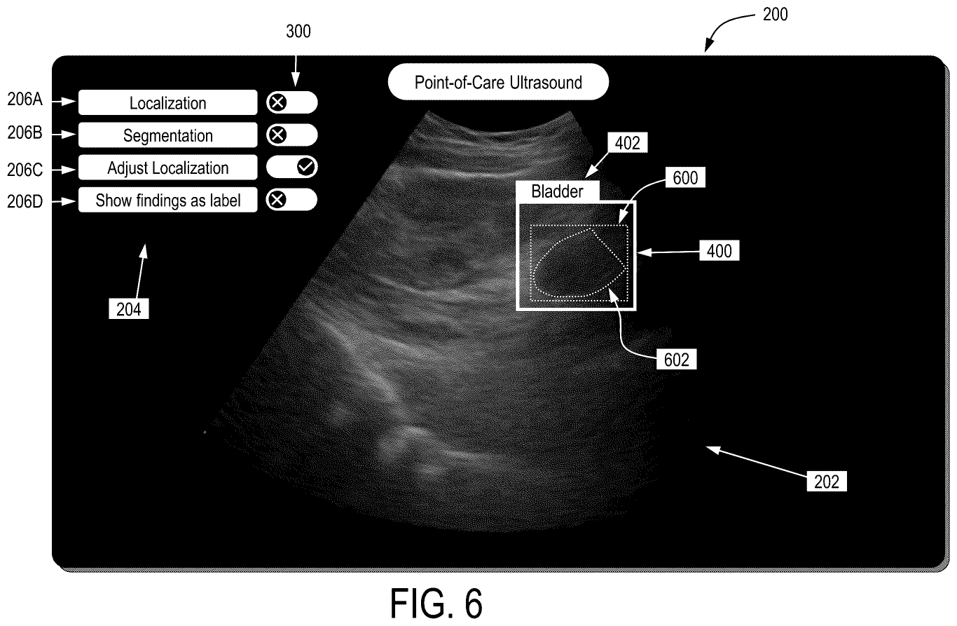

In the example of FIG. 6, an image analysis interface 200 is illustrated where the third option 206C is selected by the user 116 and the system 100 has received a user input modifying an output of one or more of the trained artificial intelligence models. More specifically, as shown in FIG. 6, an object localization model has been applied to the imaging data 110 and has identified a region where an anatomical feature is predicted to be located, which is represented visually by the bounding box 400. Next, the user 116 provided an input parameter as a user input via interface 200. In the example of FIG. 6, the input parameter includes an adjustment of the bounding box 400, which is represented visually as the modified bounding box 600. In embodiments, this user input (i.e., the adjusted bounding box 600) may be provided to a second trained artificial intelligence model, such as the segmentation model, for analysis (i.e., the segmentation model only needs to analyze the region identified by the adjusted localization). As a result, a different bounding polygon 602 may be output from the segmentation model identifying a more specific region where the anatomical feature is predicted to be present.

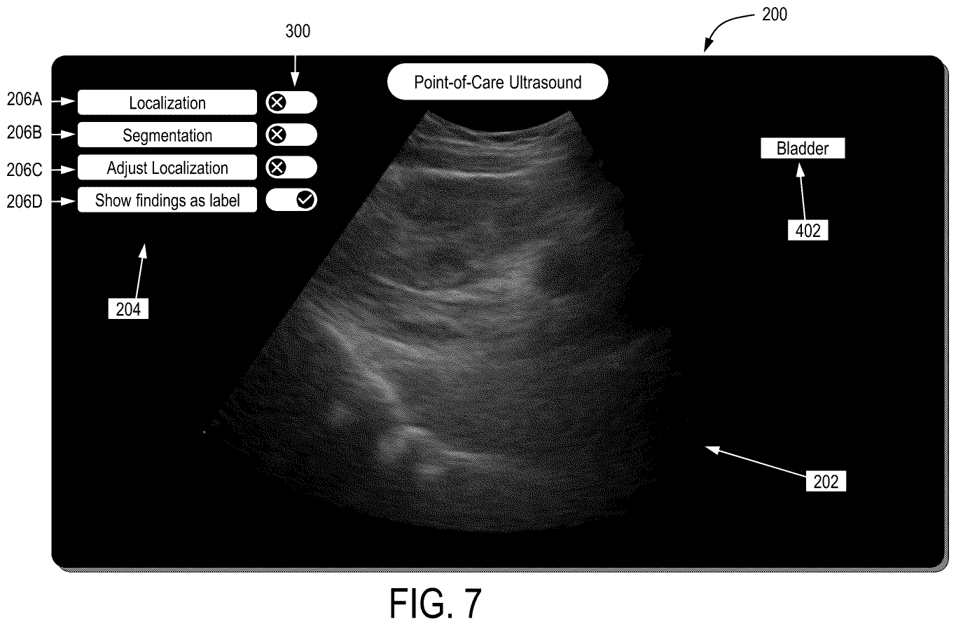

In the example of FIG. 7, an image analysis interface 200 is illustrated where the fourth option 206D is selected by the user 116 and the system 100 has displayed the output of the one or more trained artificial intelligence models as a classification label 402 only. That is, in particular embodiments, the menu 204 can include a user-selectable options 206D that suppresses or removes at least a portion of the output of one or more trained artificial intelligence models, such as the bounding boxes 400, 600 and/or bounding polygons 500, 602.

As described above, the personalized, semi-automatic feature analysis provided by the systems and methods of the present disclosure can improve the performance of the one or more trained artificial intelligence models. For example, by allowing the user 116 to modify the localization box 400, the risk of over-segmentation and/or under-segmentation can be greatly reduced. Additionally, by limiting the region in which the segmentation model is applied to, the amount of time needed to apply the one or more trained artificial intelligence models can be reduced (i.e., faster AI model inferences can be obtained). The output of the one or more trained artificial intelligence models may also become more accurate as a result of compensating for the input parameters received as user input. Further, the image analysis interface 200 can save computational power and time by allowing more experienced users to suppress or remove features (e.g., bounding boxes, polygons, etc.) that might otherwise interfere with their workflow.

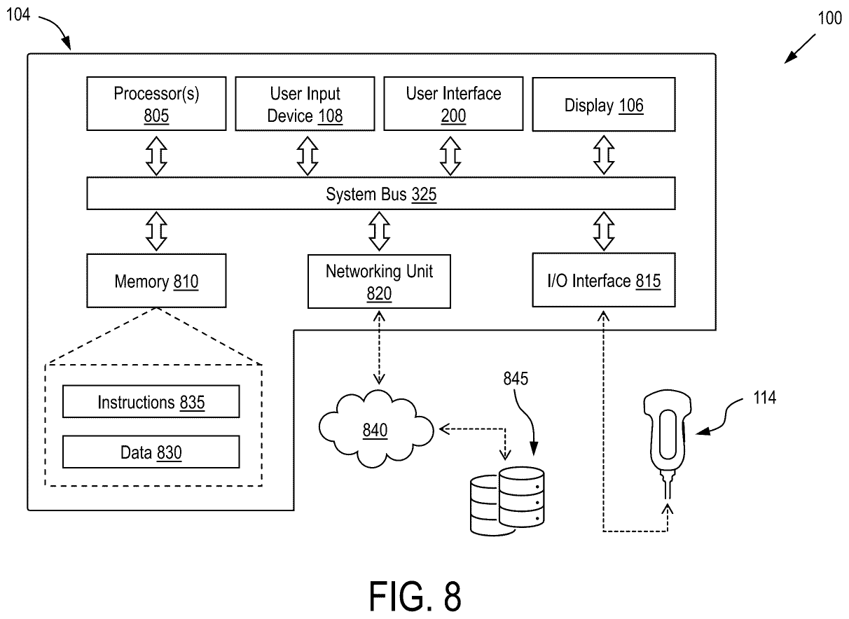

Turning now to FIG. 8, a block diagram of an imaging analysis system 100 configured to assist in the medical imaging analysis of a subject 102 is illustrated in accordance with further aspects of the present disclosure. The system 100 comprises an electronic device 104 including one or more of the following components: one or more processors 805; machine-readable memory 810; an input/output interface 815; and/or a networking unit 820. One or more of the components of the electronic device 104 may be interconnected and/or communicate through a system bus 825 containing conductive circuit pathways through which instructions (e.g., machine-readable signals) may travel to effectuate communication, tasks, storage, and the like. As described above, the electronic device 104 may also include a user input device 108, a display device 106, and a user interface 200.

The one or more processors 805 may include one or more high-speed data processors adequate to execute the program components described herein and/or perform one or more steps of the methods described herein. The one or more processors 805 may include a microprocessor, a multi-core processor, a multithreaded processor, an ultra-low voltage processor, an embedded processor, and/or the like, including combinations thereof. The one or more processors 805 may include multiple processor cores on a single die and/or may be a part of a system on a chip (SoC) in which the processor 805 and other components are formed into a single integrated circuit, or a single package. As a non-exhaustive list, the one or more processors 805 may include one or more of an Intel® Architecture Core™ based processor, such as a Quark™, an Atom™, an i3, an i5, and i7, or an MCU-class processor, an Advanced Micro Devices, Inc. (AMD) processor such as a Ryzen or Epyc based processor, an A5-A10 processor from Apple® Inc., a Snapdragon™ processor from Qualcomm® Technologies, Inc., and/or the like.

The input/output (I/O) interface 815 of the electronic device 104 may include one or more I/O ports that provide a physical connection to one or more devices, such as one or more health monitoring devices. Put another way, the I/O interface 815 may be configured to connect one or more peripheral devices of the system 100 in order to facilitate communication and/or control of between such devices. For example, the system 100 may include a medical imaging device 114 such as an ultrasound probe, which may be coupled to the electronic device 104 via the I/O interface 815. In some embodiments, the I/O interface 815 may include one or more serial ports.

The networking unit 820 of the electronic device 104 may include one or more types of networking interfaces that facilitate wireless communication between one or more components of the electronic device 104 and/or between the electronic device 104 and one or more external components. In embodiments, the networking unit 820 may operatively connect the electronic device 104 to a communications network (840), which may include a direct interconnection, the Internet, a local area network (“LAN”), a metropolitan area network (“MAN”), a wide area network (“WAN”), a wired or Ethernet connection, a wireless connection, a cellular network, and similar types of communications networks, including combinations thereof. In some examples, electronic device 104 may communicate with one or more remote data storage repositories 845, including but not limited to remote/cloud-based servers, cloud-based services, and/or wireless devices via the networking unit 820.

The memory 810 of the electronic device 104 can be variously embodied in one or more forms of machine-accessible and machine-readable memory. In some examples, the memory 810 may include one or more types of memory, including one or more types of transitory and/or non-transitory memory. In particular embodiments, the memory 810 may include a magnetic disk storage device, an optical disk storage device, an array of storage devices, a solid-state memory device, and/or the like, including combinations thereof. The memory 810 may also include one or more other types of memory, such as dynamic random-access memory (DRAM), static random-access memory (SRAM), erasable programmable read-only memory (EPROM), electrically erasable programmable read-only memory (EEPROM), Flash memory, and/or the like.

In particular embodiments, the memory 810 can be configured to store data 830 and machine-readable instructions 835 that, when executed by the one or more processors 805, cause the electronic device 104 to perform one or more steps of the methods and/or processes described herein. Put another way, provided herein is a computer-readable storage medium 810 having stored thereon machine-readable instructions 835 to be executed by one or more processors 805, and one or more processors 805 configured by the machine-readable instructions 835 stored on the computer-readable storage medium 810 to perform one or more of the operations of the methods described herein.

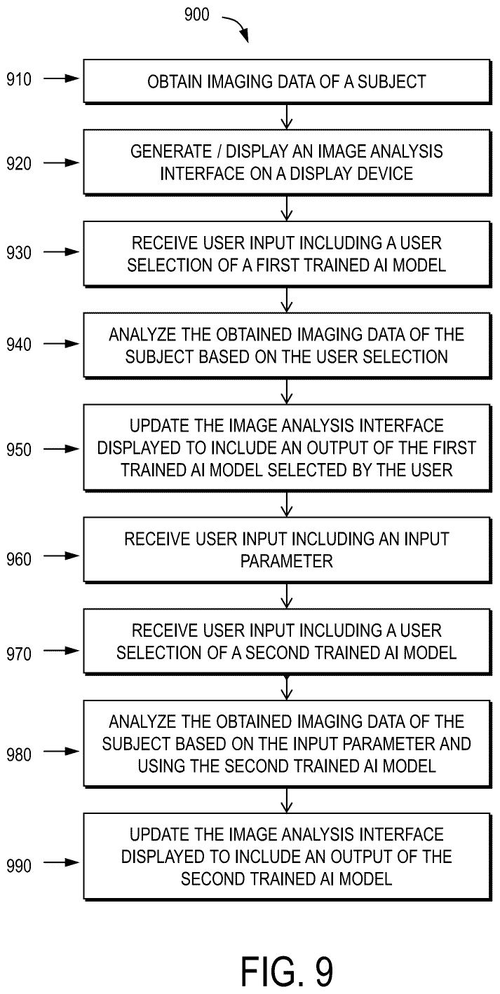

Also provided herein are computer-implemented methods for personalized, semi-automatic feature analysis using a medical imaging system 100 comprising a medical imaging device 114 and an electronic device 104 as described above. For example, with reference to FIG. 9, a computer-implemented method 900 for personalized, semi-automatic feature analysis of medical imaging data is illustrated in accordance with certain aspects of the present disclosure. In embodiments, the method 900 can include: in a step 910, obtaining imaging data of a subject; in a step 920, displaying an image analysis interface on a display device of an electronic device; in a step 930, receiving user input including a selection of at least a first trained artificial intelligence models; in a step 940, analyzing the obtained imaging data using the first trained artificial intelligence model in response to receiving the user selection; in a step 950, updating the image analysis interface to include an output of at least the first trained artificial intelligence model selected by the user; in a step 960, receiving user input including an input parameter used to modify the operation of one or more trained artificial intelligence models; in a step 970, receiving user input including a selection of at least a second trained artificial intelligence models; in a step 980, analyzing the obtained imaging data using the second trained artificial intelligence model in response to receiving the user selection from step 970, wherein the operation of the second trained artificial intelligence model is modified based on the input parameter received as user input; and in a step 990, updating the image analysis interface to include an output of at least the second trained artificial intelligence model.

More specifically, step 910 of the method 900 can include obtaining imaging data 110 of a subject 102. In particular embodiments, imaging data 110 may be obtained from a medical imaging device 114, such as an ultrasound machine, an MRI machine, a CT machine, and/or the like. Put another way, the imaging data 110 may be obtained in real-time relative to the analytical operations discussed below. In further embodiments, the imaging data 110 may be obtained from a memory (e.g., memory 810) and/or a remote server 845 storing data collected during a previous period of time.

In embodiments, step 920 of the method 900 can include generating and/or displaying an image analysis interface 200 on a display device 106 of an electronic device 104. In particular embodiments, the image analysis interface 200 can include one or more medical images 202 generated based on the obtained imaging data 110, as well as a menu 204 of user-selectable options 206.

In embodiments, step 930 of the method 900 can include receiving, via a user input device 108 of an electronic device 104, user input that includes at least a selection of at least a first trained artificial intelligence model. That is, the user input can include an instruction to analyze the received imaging data 110 using at least a first artificial intelligence model. In some embodiments, the first artificial intelligence model can be an object localization model trained to identify and localize a plurality of anatomical features within the imaging data 110. In particular embodiments, the user input may be received as a selection of one or more predefined options 206A-F displayed as part of the menu 204 of user-selectable options 206.

In embodiments, step 940 of the method 900 can include analyzing the obtained imaging data 110 using at least the first trained artificial intelligence model in response to receiving the user selection of at least the first trained artificial intelligence model. Put another way, the step 940 can include applying one or more trained artificial intelligence models to the imaging data 110 based on the user input received. As described above, at least one of the artificial intelligence models used to analyze the imaging data 110 can include an object localization model.

In embodiments, step 950 of the method 900 can include updating the image analysis interface 200 displayed on the display device 106 to include an output of at least the first trained artificial intelligence model. In particular embodiments, the output can include one or more of: (1) a bounding box 400 identifying a region within at least one medical image 202 generated based on the imaging data 110 received, wherein the region contains an anatomical feature of the subject 102 identified by one or more of the trained artificial intelligence models; (2) a bounding polygon 500, 602 segmenting an anatomical feature within at least one medical image 202 generated based on the imaging data 110 received, wherein the segmented anatomical feature was identified by one or more of the trained artificial intelligence models; and/or (3) a classification of an anatomical feature within at least one medical image 202 generated based on the imaging data 110 received, wherein the anatomical feature was identified by one or more of the trained artificial intelligence models.

In embodiments, step 960 of the method 900 can include receiving, via a user input device 108 of an electronic device 104, user input that includes at least an input parameter to be used to modify the subsequent operation of one or more trained artificial intelligence models. In particular embodiments, the input parameter can include an adjustment to the output of a previously executed artificial intelligence model. For example, in some embodiments, the output of the first artificial intelligence model applied in the step 940 may include a bounding box 400, and the input parameter can include an adjustment to the size and/or relative shape of the bounding box (e.g., adjusted bounding box 600). As such, the user input can include an instruction to modify the localization of at least one anatomical feature identified by the first artificial intelligence model.

In embodiments, step 970 of the method 900 can include receiving, via a user input device 108 of an electronic device 104, user input that includes at least a selection of at least a second trained artificial intelligence model. That is, the user input can include an instruction to analyze the received imaging data 110 using at least a second artificial intelligence model. In some embodiments, the second artificial intelligence model can be a feature segmentation model trained to identify, localize, and segment a plurality of anatomical features within the imaging data 110. In particular embodiments, the user input may be received as a selection of one or more predefined options 206A-F displayed as part of the menu 204 of user-selectable options 206.

In embodiments, step 980 of the method 900 can include analyzing the obtained imaging data 110 using at least the second trained artificial intelligence model in response to receiving the user selection of at least the second trained artificial intelligence model. Put another way, the step 980 can include applying at least a second trained artificial intelligence model different from the first trained model to the imaging data 110 based on the user input received. As described above, the second artificial intelligence model used to analyze the imaging data 110 can include at least one feature segmentation model.

In embodiments, step 990 of the method 900 can include updating the image analysis interface 200 displayed on the display device 106 to include an output of at least the second trained artificial intelligence model. In particular embodiments, the output can include one or more of: (1) a bounding box 400 identifying a region within at least one medical image 202 generated based on the imaging data 110 received, wherein the region contains an anatomical feature of the subject 102 identified by one or more of the trained artificial intelligence models; (2) a bounding polygon 500, 602 segmenting an anatomical feature within at least one medical image 202 generated based on the imaging data 110 received, wherein the segmented anatomical feature was identified by one or more of the trained artificial intelligence models; and/or (3) a classification of an anatomical feature within at least one medical image 202 generated based on the imaging data 110 received, wherein the anatomical feature was identified by one or more of the trained artificial intelligence models.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used herein, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should also be understood that, unless clearly indicated to the contrary, in any methods claimed herein that include more than one step or act, the order of the steps or acts of the method is not necessarily limited to the order in which the steps or acts of the method are recited.

The above-described examples of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, comprising an object oriented programming language such as C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, comprising a local area network (LAN) or a wide area network (WAN), or the connection can be made to an external computer (for example, through the Internet using an Internet Service Provider). In some examples, electronic circuitry comprising, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture comprising instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A system configured to assist in the medical imaging analysis of a subject, the system comprising:
an electronic device comprising:
a display device configured to display an image analysis interface;
a user input device configured to receive user input from a user;
a computer-readable storage medium having stored thereon machine-readable instructions to be executed by one or more processors; and
one or more processors configured by the machine-readable instructions stored on the computer-readable storage medium to perform the following operations: (i) receive imaging data of the subject; (ii) generate and display on the display device an image analysis interface, wherein the image analysis interface comprises the imaging data and a menu of user-selectable options; (iii) receive user input via the user input device, wherein the user input includes a selection of one or more trained artificial intelligence models; and (iv) analyze the imaging data based on the user input received.

2. The system of claim 1, wherein the one or more trained artificial intelligence models includes at least one of a trained object localization model and a trained feature segmentation model.

3. The system of claim 1, wherein the one or more processors are further configured to receive user input via the user input device, wherein the user input includes an input parameter used to modify the operation of the one or more trained artificial intelligence models.

4. The system of claim 3, wherein the input parameter includes a region of interest within at least one medical image generated based on the received imaging data of the subject.

5. The system of claim 1, wherein image analysis interface generated by the one or more processors and displayed on the display device further comprises an output of the one or more trained artificial intelligence models.

6. The system of claim 5, wherein the output of the one or more trained artificial intelligence models includes at least one of:
a bounding box identifying a region within at least one medical image generated based on the received imaging data of the subject, wherein the region contains an anatomical feature of the subject identified by the one or more trained artificial intelligence models;
a bounding polygon segmenting an anatomical feature within at least one medical image generated based on the received imaging data of the subject, wherein the segmented anatomical feature was identified by the one or more trained artificial intelligence models; and
a classification of an anatomical feature within at least one medical image generated based on the received imaging data of the subject, wherein the anatomical feature was identified by the one or more trained artificial intelligence models.

7. The system of claim 1, wherein the menu of user-selectable options of the image analysis interface comprises:
a first option to select a first trained artificial intelligence model from the one or more trained artificial intelligence models, wherein the first trained artificial intelligence model is a trained object localization model;
a second option to select a second trained artificial intelligence model from the one or more trained artificial intelligence models, wherein the second trained artificial intelligence model is a trained feature segmentation model;
a third option to modify an output of at least one of the first and second trained artificial intelligence models; and
a fourth option to provide an input parameter used to modify the operation of at least one of the first and second trained artificial intelligence models.

8. The system of claim 1, wherein the display device is a touch-enabled display, the user input device includes the touch-enabled display, and the user input comprises touch data received via the touch-enabled display.

9. The system of claim 1, further comprising:
a medical imaging device in communication with the electronic device and configured to obtain imaging data of the subject, wherein the medical imaging device includes at least one of an ultrasound imaging device, a magnetic resonance imaging machine, and a computed tomography machine.

10. A point-of-care imaging system configured to assist in the medical imaging analysis of a subject, the system comprising:
a medical imaging device configured to obtain imaging data of the subject; and
an electronic device in communication with the medical imaging device, wherein the electronic device comprises:
a display device configured to display an image analysis interface;
a user input device configured to receive user input from a user;
a computer-readable storage medium having stored thereon machine-readable instructions to be executed by one or more processors; and
one or more processors configured by the machine-readable instructions stored on the computer-readable storage medium to perform the following operations: (i) receive imaging data of the subject from the medical imaging device; (ii) generate and display on the display device an image analysis interface, wherein the image analysis interface comprises the imaging data and a menu of user-selectable options; (iii) analyze the received imaging data using at least a first trained artificial intelligence model, wherein an output of the first trained artificial intelligence model includes a bounding box identifying a region within at least one medical image generated based on the received imaging data, and wherein the region includes an anatomical feature of the subject identified by the first trained artificial intelligence model; (iv) receive user input via the user input device (108), wherein the user input includes an input parameter used to modify the operation of at least a second trained artificial intelligence model; and (v) analyze the received imaging data using at least the second trained artificial intelligence model based on the input parameter received as user input.

11. The system of claim 10, wherein the first trained artificial intelligence model is a trained object localization model and the second trained artificial intelligence model is a trained feature segmentation model.

12. The system of claim 10, wherein the input parameter received as user input includes an adjustment of the output of the first trained artificial intelligence model.

13. A computer-implemented method for personalized, semi-automatic feature analysis using a medical imaging system including a medical imaging device and an electronic device in communication with the medical imaging device, the method comprising:
obtaining, via the medical imaging device, imaging data of a subject;
displaying, on a display device of the electronic device, an image analysis interface comprising one or more images generated based on the obtained imaging data, and a menu of user-selectable options;
receiving, via a user input device of the electronic device, user input including a selection of at least a first trained artificial intelligence model, wherein at least the first trained artificial intelligence model is selected from the menu of user-selectable options;
in response to receiving the selection of at least the first trained artificial intelligence model, analyzing the obtained imaging data using at least the first trained artificial intelligence model;
updating the image analysis interface displayed via the display device of the electronic device to include an output of at least the first trained artificial intelligence model selected from the menu of user-selectable options;
receiving, via the user input device of the electronic device, user input including an input parameter used to modify the operation of one or more trained artificial intelligence models;
receiving, via the user input device of the electronic device, user input including a selection of at least a second trained artificial intelligence model selected from the menu of user-selectable options;
in response to receiving the selection of at least the second trained artificial intelligence model, analyzing the obtained imaging data using at least the second trained artificial intelligence model and the input parameter received as user input; and
updating the image analysis interface displayed via the display device of the electronic device to include an output of at least the second trained artificial intelligence model.

14. The computer-implemented method of claim 13, wherein the input parameter received as user input includes an adjustment of the output of the first trained artificial intelligence model.

15. The computer-implemented method of claim 14, wherein the first trained artificial intelligence model is a trained object localization model, and the second trained artificial intelligence model is a trained feature segmentation model.
